(19) United States
(12) Patent Application Publication
(10) Pub. No.: US 2026/0334383 A1
(43) Pub. Date: Oct. 29, 2026

(54) VINEYARD PANEL SYSTEM AND METHOD OF CONTROLLING SAME

(71) Applicant: SOLARVINE LTD., Yeroham (IL)

(72) Inventor: Dan GOLDBLAT, Yeroham (IL)

(73) Assignee: SOLARVINE LTD., Yeroham (IL)

(21) Appl. No.: 19/107,246

(22) PCT Filed: Aug. 29, 2023
(86) PCT No.: PCT/IL2023/050920

§ 371 Date: Feb. 27, 2025

(30) Foreign Application Priority Data
Aug. 31, 2022 (IL) 296104

(51) Int. Cl. A01G 9/24; A01G 9/22; A01G 17/00; H02S 20/30

(52) U.S. Cl. CPC A01G 9/243; A01G 9/22; A01G 17/00; H02S 20/30

(57) ABSTRACT

A panel system is disclosed. The panel system comprising: one or more panel units configured to be assembled in vine rows in a vineyard, each unit comprising: a support for supporting panels; one or more panels hingedly connected to the support; and, at least one actuator configured to change a state of the one or more panels from an operative state to an inoperative state, wherein a total height and a total width of each panel unit at the inoperative state are determined based on a working space defined by the agricultural equipment.

### CROSS-REFERENCE TO RELATED APPLICATIONS

This application claims the benefit of priority of Israeli Patent Application No. 296104, filed Aug. 31, 2022, which is hereby incorporated by reference in its entirety.

### FIELD OF THE INVENTION

The present invention relates generally to panel systems. More specifically, the present invention relates to panel systems to be assembled in vineyards.

### BACKGROUND OF THE INVENTION

Panel systems, more specifically solar panel (i.e., photo voltaic (PV) panel) systems are becoming one of the most popular and reliable green energy sources. An effective solar panel field utilizes vast amounts of land, for example, 1 MW solar system will require about 1 hectare (10,000 m²). This land, if can be combined with agricultural uses, can yield both agricultural products and electricity. However, assembling solar panel fields in agricultural area, more specifically in cultivated fields is challenging, as the solar panel systems may limit the ability to use automated agricultural equipment. This is evident when assembling solar panel fields in vineyards.

Vineyards, however, may benefit from the assembly of solar panels, shade nets or other shading panels in addition to energy production. Sunlight is the primary energy source plants use in photosynthesis to convert carbon dioxide and water into sugars needed to produce and ripen grapes. Nevertheless, an excessive amount of direct sunlight radiating on the grapes during low humidity conditions leads to tissue damage commonly referred to as sunburn. Sunburn damage to table grapes generally renders them unmarketable. Other undesirable results of excessive exposure to sunlight at a vineyard are increased water demand due to a corresponding increase in evapotranspiration, or water evaporating through the soil. Furthermore, excessive amount of direct sunlight may also cause fast ripening which derogates the grapes quality as well as wine quality.

Sunburn damage can be avoided by providing adequate shading at the vineyard to decrease the amount of direct sunlight to which the grapes are exposed.

There have been some attempts to implement solar panels and/or shading panels in vineyards. These attempts resulted in the use of support structures that are supported above agricultural fields at heights that allow the passage of large mechanized farm equipment to pass beneath.

Although the shading provided by the solar panels using these support structures significantly decreases the amount of direct sunlight to which the grapes are exposed, the arrangement of such support structures restricts the efficient harvesting of grapes using an automated harvester and therefore is not practically implementable in a vineyard setting.

Therefore, an object of the present invention is to provide a vineyard panel system that allows speedy mechanized treatments (e.g., harvesting, pruning, spraying, trimming, canopy management, etc.) of the grapes.

### SUMMARY OF THE INVENTION

Some aspects of the invention are directed to a panel system, comprising: one or more panel units configured to be assembled in vine rows in a vineyard, each unit comprising: a support for supporting panels; one or more panels hingedly connected to the support; and, at least one actuator configured to change a state of the one or more panels from an operative state to an inoperative state; and a controller configured to: receive an indication related to at least one of: a location and a trajectory of agricultural equipment traveling in the vineyard; and control the at least one actuator to change the state of the one or more panels, in response to the indication, wherein the inoperative state allows the free traveling of the agricultural equipment in the vineyard.

In some embodiments, at the inoperative state the panel units allow the agricultural equipment at least one of: to harvest grapes, to trim stems, to prune, to spray, and to manage the canopy of vines in the vineyard. In some embodiments, a total height and a total width of each panel unit at the inoperative state are determined based on a working space defined by the agricultural equipment.

In some embodiments, the indication is received from a user via one of: a user device in communication with the controller, and a user interface associated with the controller. In some embodiments, the indication is received from a detector configured to detect at least one of, the location and the trajectory of the agricultural equipment in the vineyard. In some embodiments, the detector is selected from, a camera located in the vineyard, a camera located on a drone hovering above the vineyard, GPS sensors, a proximity sensor, a LIDAR, an RFID reader configured to read an RFID tag attached to the agricultural equipment, and an electrical switch.

In some embodiments, the controller is configured to: receive the indication that agricultural equipment is traveling in the vineyard toward at least one panel unit; and change the state of the at least one panel unit to the inoperative state, based on the received indication. In some embodiments, at the inoperative state the one or more panels are tilted at a rotation angle of between 0 to 40 degrees with respect to a normal to vineyard surface. In some embodiments, the panel system further comprises at least one sun radiation sensor configured to assess the sun radiation, and wherein the controller is further configured at the operative state, to control the at least one actuator according to signals form the at least one sun radiation sensor. In some embodiments, the one or more panels are selected from, solar panels and shading panels.

Some additional aspects of the invention are directed to a method of managing a panel system in a vineyard, comprising: receiving an indication related to at least one of, a location and a trajectory of agricultural equipment traveling in the vineyard; and controlling at least one actuator to change the state of one or more panels included in at least ones panel unit, in response to the indication, wherein each panel unit comprises a support for supporting panels; the one or more panels hingedly connected to the support; and, the at least one actuator configured to change a state of the one or more panels from an operative state to an inoperative state, and wherein the inoperative state allows the free traveling of the agricultural equipment in the vineyard.

In some embodiments, the indication is received from a user via one of: a user device in communication with the controller, and a user interface associated with the controller. In some embodiments, the indication is received from a detector configured to detect at least one of, the location and the trajectory of the agricultural equipment in the vineyard. In some embodiments, the detector is selected from, a camera located in the vineyard, a camera located on a drone hovering above the vineyard, GPS sensors, a proximity sensor, a LIDAR, an RFID reader configured to read an RFID tag attached to the agricultural equipment and electrical switch.

In some embodiments, the method further comprising: receiving the indication that agricultural equipment is traveling in the vineyard toward at least one panel unit; and changing the state of the at least one panel unit to the inoperative state.

In some embodiments, at the inoperative state the one or more panels are tilted at a rotation angle of between 0 to 40 degrees with respect to a normal to the vineyard surface. In some embodiments, the method further comprises receiving from at least one sun radiation sensor, a signal indicative of the sun radiation, and controlling the at least one actuator to tilt the one or more panels during the deployed state according to signals form the at least one sun radiation sensor.

Some additional aspects of the invention may be directed to a panel system, comprising: one or more panel units configured to be assembled in vine rows in a vineyard, each unit comprising: a support for supporting panels; one or more panels hingedly connected to the support; and, at least one actuator configured to change a state of the one or more panels from an operative state to an inoperative state, wherein a total height and a total width of each panel unit at the inoperative state are determined based on a working space defined by the agricultural equipment.

In some embodiments, the working space defined by the agricultural equipment has a height of at most 4000 mm and a width of at most 1000 mm. In some embodiments, the one or more panels are hingedly connected to the support at a height of at least 100 mm from the ground. In some embodiments, at folded state the panel units allow the agricultural equipment at least one of: to harvest grapes, to trim stems, to prune, to spray, and to manage canopy of vines in the vineyard.

In some embodiments, the panel system further comprises: a controller configured to: receive an indication related to one of, a location and a trajectory of agricultural equipment traveling in the vineyard; and control the at least one actuator to change the state of the one or more panels, in response to the indication. In some embodiments, the indication is received from a user via one of: a user device in communication with the controller, and a user interface associated with the controller.

In some embodiments, the indication is received from a detector configured to detect at least one of, the location and the trajectory of the agricultural equipment in the vineyard. In some embodiments, the detector is selected from, a camera located in the vineyard, a camera located on a drone hovering above the vineyard, GPS sensors, a proximity sensor, a LIDAR, an RFID reader configured to read an RFID tag attached to the agricultural equipment, and a simple switch.

In some embodiments, the controller is configured to: receive the indication that agricultural equipment is traveling in the vineyard toward at least one panel unit; and change the state of the at least one panel unit to the inoperative state. In some embodiments, at the inoperative state the one or more panels are tilted at a rotation angle of between 1 to 40 degrees with respect to a normal to the vineyard surface. In some embodiments, the panel system further comprises at least one sun radiation sensor configured to assess the sun radiation, and wherein the controller is further configured at a deployed state to control the at least one actuator according to signals form the at least one sun radiation sensor. In some embodiments, the one or more panels are selected from, solar panels and shading panels.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is an illustration of a panel unit assembled in a vine row according to some embodiments of the invention;

FIG. 1B is an illustration of a panel unit in an inoperative state inside a working space defined by an agricultural equipment according to some embodiments of the invention;

FIGS. 2 and 3 are illustrations of panel systems, during the deployment of two different agricultural equipment according to some embodiments of the invention;

FIG. 4 is a block diagram of the panel system according to some embodiments of the invention;

FIG. 5 is a flowchart of a method of controlling the panel system according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

Some aspects of the invention may be directed to panel systems that include a plurality of panel units configured to be assembled in vine rows in a vineyard. Each panel unit may have at least two states, an inoperative state, and a deployed state. At the inoperative state each panel unit allows free traveling of any agricultural equipment in the vineyard (e.g., a harvester, a trimmer, and the like) while conducting one of, harvesting grapes, trimming stems, pruning, spraying, and canopy managing.

At the deployed state each panel unit is configured to provide shadow to the vines beneath the panels and/or produce electricity (when the panels are solar panels).

Such a panel system may allow the advancement of automated agricultural equipment such as a grape harvester through the clearance between adjacent rows of grapevines, defined as inter-row clearance. The system may include a shading promoting panel that may be mounted onto a horizontal rigid elongated element extending between adjacent posts that have been anchored at the same side of a grapevine row. In an operative state, when the panel is found to laterally protrude into the clearance between adjacent rows of grape vines and to interfere with the advancing grape harvester, the supporting elongated element is suitably rotated to cause the panel to pivot or otherwise be angularly displaced until the protruding dimension of the panel is sufficiently reduced to allow unrestrained passage of the grape harvester between adjacent rows of grape vines.

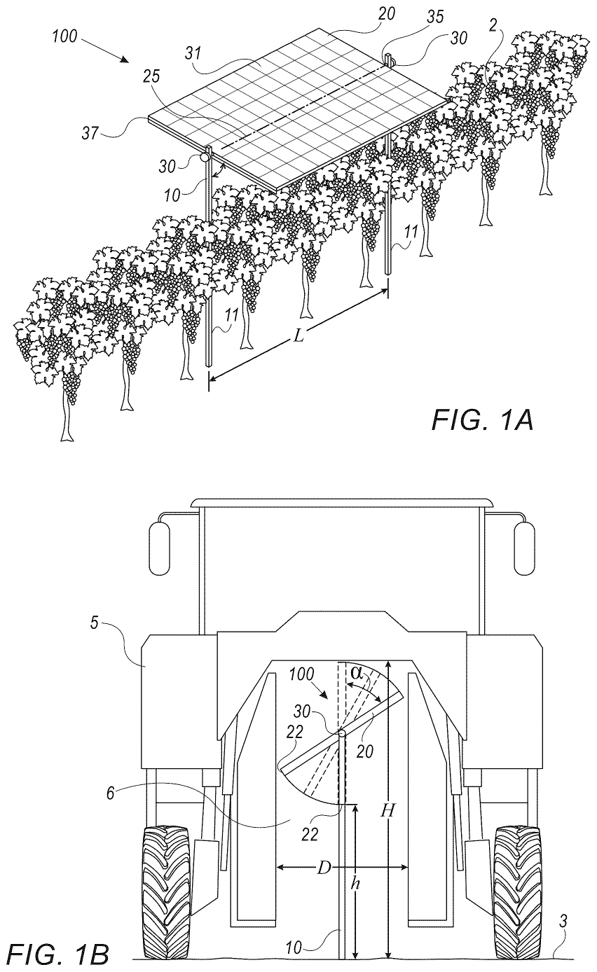

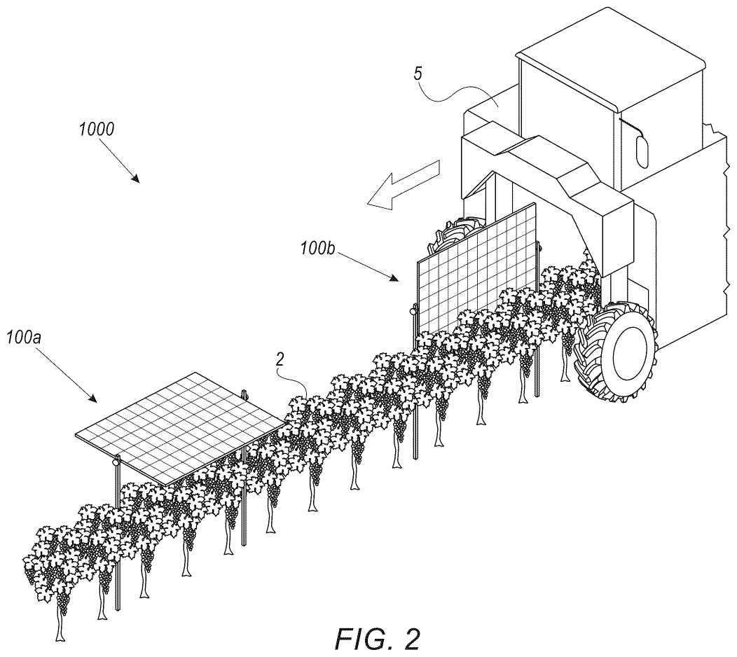

Reference is now made to FIG. 1A which is an illustration of a panel unit assembled in a vine row according to some embodiments of the invention. A panel unit 100 may include a support 10 for supporting panels, one or more panels 20 hingedly connected to support 10 and at least one actuator 30 configured to change a state of the one or more panels from a deployed/operative state to an inoperative state.

In some embodiments, support 10 may include two longitudinally spaced posts 11, spaced along the long dimension L of a vineyard row 2, which are secured within the ground underlying vineyard row 2 at a boundary region thereof proximate to an inter-row clearance between neighboring vineyard rows. In a non-limiting example, posts 11 are made of aluminum due to its light weight and high strength, or steal, although other materials may be used as well. In some embodiments, posts 11 are lighter and consequently thinner than the of pillars extending downwardly from a prior art solar panel support structure, the posts are advantageously able to be secured within the ground underlying vineyard row 2 without interfering with the vines, rather than being secured within the ground underlying inter-row clearance as practiced in the prior art due to the significantly larger thickness of the pillars.

In some embodiments, support 10 may be or may include the trellising system of the vineyard. In such case one or more panels 20 may be hingedly connected to the trellising system via at least one actuator 30 configured to change a state of the one or more panels from a deployed/operative state to an inoperative state. The trellising system may any known trellising system, such as, High Cordon (HC), Umbrella Kniffen, or Geneva Double Curtain (GDC), Vertical Shoot Positioning (VSP) and the like.

One or more panels 20 may include any panel/structure/shade net that can provide shade. One or more panels 20 may include one or more solar panels and/or shading panels. For example, panels 20 may include one or more solar panels 31 mounted on a frame 37, as illustrated. In another example, panels 20 may include one or more shading panels made from one or more rigid substantially opaque surfaces mounted on frame 37, for providing shading. In yet another example, panel 20 may include a shading panel made from a substantially opaque flexible sheet (e.g., a shading net, a polymeric sheet, etc.) starched over a frame, such as frame 37.

In another nonlimiting example, frame 37 of panel 31 for generating electricity may be rearwardly supported, e.g., at the shade-able side thereof, facing away from the solar cells, by two longitudinally spaced, shortened rectangular mounting bars in attached abutting relation, such as by welding, with frame 37. The mounting bars may be perpendicularly attached such as by welding to a rail, e.g. a rectilinear rail (not shown), longitudinally extending between the two posts 11 and preferably aligned with a centerline of panel 31. Although not shown, additional solar panels may be mounted to additional mounting bars that may be attached to the same rail.

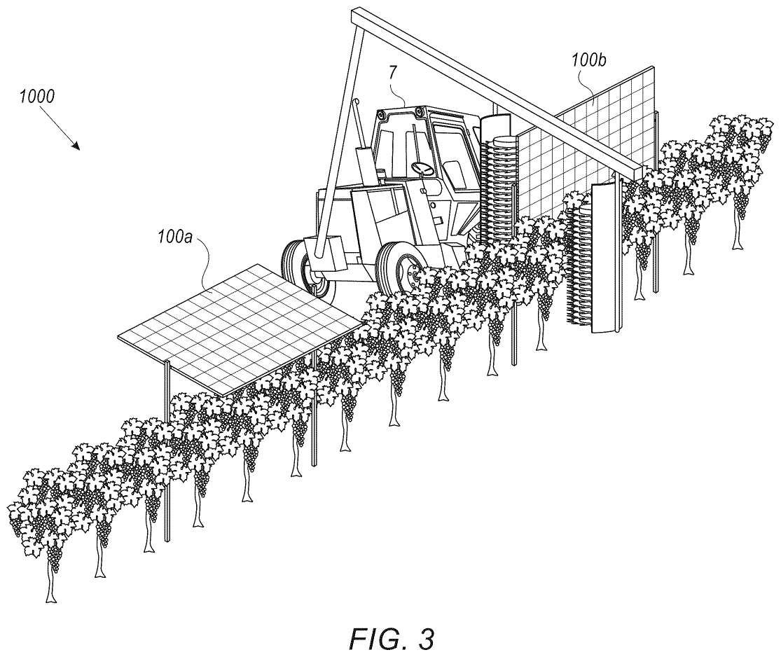

At least one actuator 30 may be connected to an axis of a connector 35 connecting one or more panels 20 to support 10. One or more panels 20 may be hingedly connected to support 10, by a rail, a shaft, a hinge, or any other connection means that may allow one or more panels 20 to rotate around a longitudinal axis 25 (parallel to vine row) of each panel, for example, the central longitudinal axis. In some embodiments, actuator 30 is configured to change a state of one or more panels 30 (e.g., by rotating the panels) from a deployed (operative) state to an inoperative state, as illustrated and discussed with respect to FIGS. 2 and 3.

At least one actuator 30 may include a motor (e.g., an electric motor) axially connected to the axis of connector 35. In some embodiments, one actuator 30 may further include a gear (e.g., a worm gear, bevel gears, etc.) axially connected between the axis of connector 35 and the axis of the motor. In some embodiments, at least one actuator 30 may be powered by other means such as pneumatic or hydraulic systems comprising pistons, and pressurized gas tanks, pumps etc.) As should be understood by one skilled in the art, at least one actuator 30 may include any mechanical or electrotechnical component that can provide an axial movement to panels 20.

Reference is now made to FIG. 1B which is an illustration of a panel unit in an inoperative state inside a working space defined by an agricultural equipment according to some embodiments of the invention. When agricultural equipment 5, e.g., harvester 5, is traveling and optionally harvesting grapes along row 2, one or more panels 20 of unit 100 may be rotated into an inoperative position that allows the free traveling of the agricultural equipment. In some embodiments, agricultural equipment 5 may be any automatic, semi-automatic, or autonomous agricultural equipment for treating vineyards. The agricultural equipment can be manually operated, automatically operated and manually driven, or completely autonomous.

Therefore, a total height H and a total width D of each panel unit 100 at the inoperative state may be determined based on a working space 6 defined by agricultural equipment 5. In some embodiments, working space 6 defined by agricultural equipment 5 has a height H of at most 4000 mm, for example, at most 3800 mm, 3500 mm, 3300 mm, 3000 mm, 2800 mm, 2500 mm, 2000 mm, and any value in between.

In some embodiments, the working space defined by the agricultural equipment has a width of at most 1000 mm, for example, at most 900 mm, at most 800 mm, at most 700 mm, at most 600 mm, at most 500 mm, and any value in between.

In some embodiments, one or more panels 20 are hingedly connected to the support 30 at a height h of at least 600 mm from a ground 3 of the vineyard, for example, an edge 22 of a panel 20, at the inoperative position may be, at least 700 mm from ground 3. at least 800 mm from ground 3, at least 900 mm from ground 3, at least 1000 mm from ground 3, at least 1100 mm, at least 1200 mm, at least 1300 mm, at least 1400 mm, at least 1500 mm, at least 1600 mm, at least 1700 mm, at least 1800 mm from ground 3 and any value in between.

In some embodiments, at the inoperative state the one or more panels are tilted at a rotation angle a of between 0 to 40 degrees with respect to a normal to vineyard ground 3. For example, at the inoperative state the one or more panels are tilted at rotation angle a of between 0 to 5 degrees, between 1 to 10 degrees, between 5 to 15 degrees, between 10 to 20 degrees, between 15 to 25 degrees, between 20 to 30 degrees, between 25 to 35 degrees, between 30 to 38 degrees, between 30 to 40 degrees, between 0 and 40 degrees, and any value or any range in between.

Reference is now made to FIG. 2 which shows a panel system 1000 comprising two or more panel units 100a and 100b at inoperative (unit 100a) and deployed/operative (unit 100b) states. Unit 100a and 100b may include substantially the same components as unit 100. In the inoperative position, agricultural equipment 5 (e.g., a harvester) can freely travel and harvest the grapes in row 2. Since a distance (e.g., height h of at least 1000 mm from ground 3) is kept between ground 3 and edge 22 (illustrated in FIG. 1B), the harvester can harvest grapes growing at heights lower than h, for example, when h=1500 mm, harvester 5 may harvest grapes at any height lower than h, e.g., between 700-1400 mm, while traveling over unit 100b as illustrated.

At a deployed (operative) state panel unit 100a may provide at least one of, shade to the vines located beneath panel unit 100a and/or electricity, when panel unit 100a comprises solar panels.

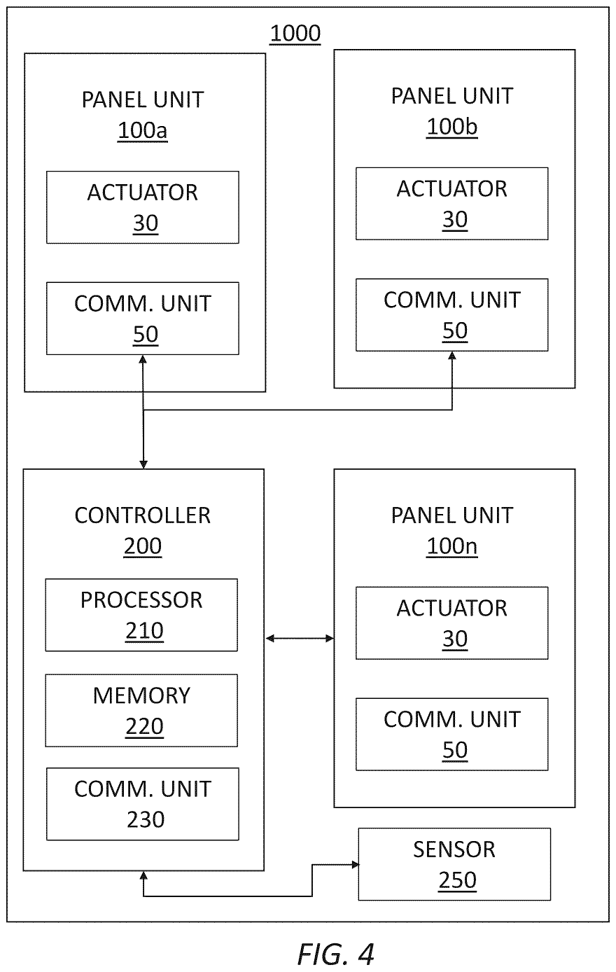

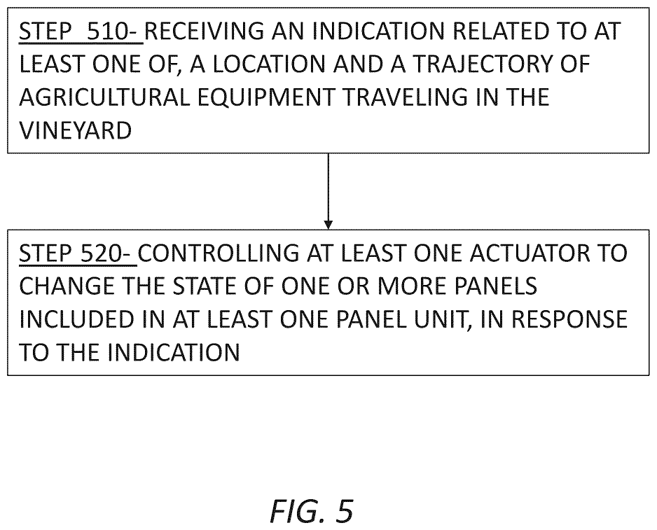

In some embodiments, system 1000 may further include a controller (illustrated in FIG. 4) configured to control actuators 30 to change the state of the one or more panels 20 from inoperative to deployed (operative) state and vise versa, for example, in response to an indication related to one or more of, a location and a trajectory of agricultural equipment traveling in the vineyard, as discussed with respect to FIGS. 4 and 5 hereinbelow.

Reference is now made to FIG. 3 which shows a panel system 1000 comprising two or more panel units 100a and 100b at an inoperative state (unit 100a) and deployed (operative) state (unit 100b). In the inoperative state, another agricultural equipment 7 (e.g., a rotary trimmer) can freely travel and trim vines in row 2. The change in states of units 100a and 100b of system 1000 in the presence of the trimmer are substantially the same as in the presence of the harvester (agricultural equipment 5) discussed with respect to FIG. 2.

Reference is now made to FIG. 4 which is a block diagram of system 1000 according to some embodiments of the invention. A system 1000 may include one or more panel units 100a, 100b .... 100n (wherein n is an integer>2) and a controller 200. In some embodiments, two or more panel units 100a, 100b .... 100n may be assembled in vine rows in a vineyard as illustrated in Figs, 2 and 3. Two or more panel units 100a, 100b .... 100n may include substantially the same components as panel unit 100 discussed above. In some embodiments, panel units 100a, 100b .... 100n may further include a communication unit 50 for communicating with controller 200, thereby transferring instructions from controller 200 to actuators 30 of each panel unit.

Communication unit 50 may include any type of communication module, either wired or wireless communication module. For example, communication unit 50 may include a wireless communication module (e.g., a Wi-Fi unit, a cellular modem, etc.) and an antenna for receiving and transmitting wireless transmission using radiofrequency waves. In another example, communication 50 may include a wired modem and at least some of panel units 100a, 100b .... 100n may be connected to controller 200 by wires (e.g., running underground or above ground in the vineyard).

In some embodiments, system 1000 may further include cables and/or wires (not illustrated) for at least one of: providing electricity to actuators 30 and to communication units 50, communicating between controller 200 and actuators 30, and collecting electricity from panels 20, when panels 20 are solar panels (e.g., PV panels). The cables may be buried underground or may be above ground.

Controller 200 may include a processor 210 that may be, for example, a central processing unit (CPU) processor, a chip or any suitable computing or computational device. Processor 210 (or one or more processors, possibly across multiple units or devices) may be configured to carry out methods described herein, and/or to execute or act as the various modules, units, etc. More than one computing controller 200 may be included system 1000 according to embodiments of the invention.

Controller 200 may include a memory 220 that may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 220 may be or may include a plurality of possibly different memory units. Memory 220 may be a computer or processor non-transitory readable medium, or a computer non-transitory storage medium, e.g., a RAM. In one embodiment, a non-transitory storage medium such as memory 220, a hard disk drive, another storage device, etc. may store instructions or code which when executed by a processor may cause the processor to carry out methods as described herein, for example, methods of managing a panel system in a vineyard.

Memory 200 be stored thereon, at least one of, an operating system, an executable code, and a database according to some embodiments of the invention.

Controller 150 may further include a communication unit 230 that may include one or more input and output devices. For example, the input devices may be or may include any suitable input devices, components or systems, e.g., a detachable keyboard or keypad, a mouse and the like. In another example, the output devices may include one or more (possibly detachable) displays or monitors, speakers and/or any other suitable output devices. Any applicable input/output (I/O) devices may be connected to controller 200. For example, a wired or wireless network interface card (NIC), a universal serial bus (USB) device or external hard drive may be included in the input devices and/or the output devices.

System 1000 may further include at least one detector 250. Sensor 250 may be configured to provide an indication related to at least one of: a location and a trajectory of agricultural equipment traveling in the vineyard. In some embodiments, sensor 250 may be selected from a camera located in the vineyard, a camera located on a drone hovering above the vineyard, Global Positioning System (GPS) detectors, a proximity detector, a Light Detection And Ranging (LIDAR) detector, a Radio Frequency Identification (RFID) reader configured to read an RFID tag attached to the agricultural equipment, a simple switch and the like.

In some embodiments, system 1000 may include additional one or more sensors, for example, temperature sensor, wind sensor, precipitation detector, pressure sensor, and the like. In some embodiments, controller 200 may receive meteorological data from external sources, such as, external sensors, external databases, the internet and the like.

Reference is now made to FIG. 5 which is a flowchart of a method of managing a panel system in a vineyard according to some embodiments of the invention. The method of FIG. 5 may be executed by controller 200 of system 1000 or by any other suitable processor.

In step 510, controller 200 may receive an indication related to at least one of: a location and a trajectory of agricultural equipment traveling in the vineyard. In some embodiments, the indication is received from a user via one of: a user device in communication with the controller, and a user interface associated with the controller. For example, an operator operating agricultural equipment 5 (e.g., harvester) may send from his/her mobile phone an alert to controller 200 (via communication unit 230) that the harvester entered the first row in the vineyard. Alternatively, an application on the operator mobile phone may use its GPS detector to continuously identify the location and trajectory of agricultural equipment 5.

Additionally or alternatively the indication may be received from a detector configured to detect at least one of, the location and the trajectory of the agricultural equipment in the vineyard. For example, sensor 250 may send the indication to controller 200, that agricultural equipment 5 or 7) entered the vineyard, 2) start treating a specific vine row, 2 approaching a specific panel unit and the like. For example, sensor 250 may be a video camera located on agricultural equipment 5 or 7 that may receive images from the surrounding of agricultural equipment 5 or 7 and determine the location and/or trajectory of the agricultural equipment based on the images. In another example, sensor 250 may be a GPS sensor attached to the agricultural equipment configured to identify temporal coordinates of agricultural equipment 5 or 7 and determine the location and/or trajectory of the agricultural equipment based on the temporal coordinates. In other examples, sensor 250 may be a proximity sensor, a LIDAR, an RFID reader configured to read an RFID tag attached to the agricultural equipment and the like.

In step 520, controller 200 may control the at least one actuator to change the state of the one or more panels, in response to the indication. In some embodiments, controller 200 may control actuator 30 to rotate one or more panels 20 to one of two states, an inoperative state and a deployed state. At a deployed state (illustrated for unit 100a in FIGS. 2 and 3) one or more panels 20 shade the vines beneath the panels and can also produce electricity, if one or more panels 20 are solar panels. In some embodiments, controller 200 may further change the angle (e.g., angle α) of the panels with respect to a normal to vineyard ground 3, in response to the direction of the radiation from the sun. For example, the angle may change during the day in order to follow the movement of the sun in the sky (e.g., based on astronomical data) in order to increase the amount of shading and/or increase electricity production. Additionally or alternatively, system 1000 may further include at least one sun radiation sensor (not illustrated) configured to assess the sun radiation, and controller 200 is further configured at a deployed state to control the at least one actuator according to signals form the at least one sun radiation sensor.

Additionally or alternatively, controller 200 may control actuator 30 based on meteorological data, for example, wind conditions, precipitation conditions (rain, snow, hail, etc.), temperature, and the like. Controller 200 may receive the information from an external database, external sensor, a weather forecast site on the internet, or from one or more sensors included in system 1000. For example, controller 200 may control all actuators 30 to rotate the panels when hail is falling, to avoid harm to the solar panels or tearing of the shading nets.

In some embodiments, when controller 200 receives an indication that agricultural equipment 5 or 7 is approaching it may control actuator 30 to rotate one or more panels 20 to the inoperative state. In the inoperative state, the panels are rotated in an angle a, with respect to the normal to the vineyard ground, to allow the free traveling of the agricultural equipment in the vineyard. In some embodiments, at inoperative state the one or more panels can be tilted at a rotation angle of between 0 to 40 degrees with respect to a normal to vineyard surface. For example, controller 200 may receive an indication that agricultural equipment 5 or 7 is approaching the vineyard and may control all actuators 30 to rotate all panels 20 of all panel units assembled in the vineyard into inoperative state. In another example, controller 200 may receive an indication that agricultural equipment 5 or 7 is approaching a specific row of vines and may control all actuators 30 to rotate all panels 20 of all panel units assembled in the row into inoperative state. In yet another example, controller 200 may receive an indication that agricultural equipment 5 or 7 is approaching a specific panel unit (e.g., unit 100b illustrated in FIGS. 2 and 3) and may control all actuator 30 to rotate all panels 20 of panel unit 100b to the inoperative state, keeping all other panel units in deployed (operative) state.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A panel system, comprising:
one or more panel units configured to be assembled in vine rows in a vineyard, each unit comprising:
a support for supporting panels;
one or more panels hingedly connected to the support; and,
at least one actuator configured to change a state of the one or more panels from an operative state to an inoperative state; and
a controller configured to:
receive an indication related to at least one of: a location and a trajectory of agricultural equipment traveling in the vineyard; and
control the at least one actuator to change the state of the one or more panels, in response to the indication,
wherein the inoperative state allows the free traveling of the agricultural equipment in the vineyard.

2. The panel system of claim 1, wherein at the inoperative state the panel units allow the agricultural equipment at least one of: to harvest grapes, to trim stems, to prune, to spray, and to manage the canopy of vines in the vineyard.

3. The panel system of claim 2, wherein a total height and a total width of each panel unit at the inoperative state are determined based on a working space defined by the agricultural equipment.

4. The panel system of claim 1, wherein the indication is received from a user via one of: a user device in communication with the controller, and a user interface associated with the controller.

5. The panel system of claim 1, wherein the indication is received from a detector configured to detect at least one of, the location and the trajectory of the agricultural equipment in the vineyard.

6. The panel system of claim 5, wherein the detector is selected from, a camera located in the vineyard, a camera located on a drone hovering above the vineyard, GPS sensors, a proximity sensor, a LIDAR, an RFID reader configured to read an RFID tag attached to the agricultural equipment, and an electrical switch.

7. The panel system of claim 1, wherein the controller is configured to:
receive the indication that agricultural equipment is traveling in the vineyard toward at least one panel unit; and
change the state of the at least one panel unit to the inoperative state, based on the received indication.

8. The panel system of claim 1, wherein at the inoperative state the one or more panels are tilted at a rotation angle of between 0 to 40 degrees with respect to a normal to vineyard surface.

9. The panel system of claim 1, further comprising at least one sun radiation sensor configured to assess the sun radiation, and wherein the controller is further configured at the operative state, to control the at least one actuator according to signals form the at least one sun radiation sensor.

10. The panel system of claim 1, wherein the one or more panels are selected from solar panels and shading panels.

11. A method of managing a panel system in a vineyard, comprising:
receiving an indication related to at least one of, a location and a trajectory of agricultural equipment traveling in the vineyard; and
controlling at least one actuator to change the state of one or more panels included in at least one's panel unit, in response to the indication,
wherein each panel unit comprises
a support for supporting panels;
the one or more panels hingedly connected to the support; and,
the at least one actuator configured to change a state of the one or more panels from an operative state to an inoperative state,
and wherein the inoperative state allows the free traveling of the agricultural equipment in the vineyard.

12. The method of claim 11, wherein the indication is received from a user via one of: a user device in communication with the controller, and a user interface associated with the controller.

13. The method of claim 11, wherein the indication is received from a detector configured to detect at least one of, the location and the trajectory of the agricultural equipment in the vineyard.

14. The method of claim 13, wherein the detector is selected from, a camera located in the vineyard, a camera located on a drone hovering above the vineyard, GPS sensors, a proximity sensor, a LIDAR, an RFID reader configured to read an RFID tag attached to the agricultural equipment and electrical switch.

15. The method of claim 11, further configured to:
receiving the indication that agricultural equipment is traveling in the vineyard toward at least one panel unit; and
changing the state of the at least one panel unit to the inoperative state.

16. The method of claim 11, wherein at the inoperative state the one or more panels are tilted at a rotation angle of between 0 to 40 degrees with respect to a normal to the vineyard surface.

17. The method of claim 11, further comprising receiving from at least one sun radiation sensor, a signal indicative of the sun radiation, and controlling the at least one actuator to tilt the one or more panels during the deployed state according to signals form the at least one sun radiation sensor.

18. A panel system, comprising:
one or more panel units configured to be assembled in vine rows in a vineyard, each unit comprising:
a support for supporting panels;
one or more panels hingedly connected to the support; and,
at least one actuator configured to change a state of the one or more panels from an operative state to an inoperative state,
wherein a total height and a total width of each panel unit at the inoperative state are determined based on a working space defined by the agricultural equipment.

19. The panel system of claim 18, wherein the working space defined by the agricultural equipment has a height of at most 4000 mm and a width of at most 1000 mm.

20. The panel system of claim 18, wherein the one or more panels are hingedly connected to the support at a height of at least 100 mm from the ground.

21.-29 (canceled)
